(19) United States
(12) Patent Application Publication
(10) Pub. No.: US 2026/0007866 A1
(43) Pub. Date: Jan. 8, 2026

(54) COMPOSITE MEDICAL BALLOON AND RELATED METHODS

(71) Applicant: BARD PERIPHERAL VASCULAR, INC., Franklin Lakes, NJ (US)

(72) Inventors: Zachary Willis, Mesa, AZ (US); Kyle Thomas, Phoenix, AZ (US); Nicholas Peterson, Mesa, AZ (US)

(21) Appl. No.: 18/880,269

(22) PCT Filed: Jun. 30, 2022
(86) PCT No.: PCT/US2022/035657

§ 371 Date: Dec. 30, 2024

(51) Int. Cl. A61M 25/10

(52) U.S. Cl. CPC A61M 25/1029; A61M 25/1034; A61M 2025/1075

(57) ABSTRACT

An apparatus 10 for performing a medical procedure includes a co-extruded inflatable balloon 13 having an outer layer 13a and an inner layer 13b comprising different materials and a shaft 11 comprising a material compatible the inner layer of the balloon and welded thereto. An inflatable balloon is also provided having an outer layer comprising a polyester and an inner layer comprising a polyamide, as is an apparatus combining an inflatable balloon comprising a polyester and a shaft comprising a polyester connected to the inflatable balloon. Related methods are also disclosed.

### BACKGROUND

Medical balloons are widely used in medical procedures. Typically, an uninflated medical balloon is inserted via an associated catheter into a body space, such as a blood vessel. When the medical balloon is inflated, the volume of the medical balloon expands, and the body space is similarly expanded. In procedures such as percutaneous transluminal angioplasty (PTA), the inflated medical balloon may be used to restore patentcy to a collapsed or blocked vessel, or to provide a treatment, such as a drug or stent, to an endovascular location.

Many current balloon catheters use dissimilar materials between the balloon and catheter, such as a PEBAX or nylon shaft as the catheter and a polyethylene terephthalate (PET) balloon, as one example. While desirable for providing the balloon with certain properties, such as enhanced strength/resistance, or possibly to achieve an affinity for a coating or drug in the case of the balloon specifically, this material difference precludes the successful use of thermal bonding operations during assembly. Instead, adhesives are used to join the balloon to the catheter shaft, but the application of adhesives increases the complexity and cost of manufacture. Furthermore, adhesive joints are often less flexible, and have a tendency to leak. Such joints are also much more difficult to validate when compared with welded joints.

Accordingly, a need is identified for a composite medical balloon that overcomes any or all of the foregoing limitations and possibly others that have yet to be identified.

### SUMMARY

An object of the disclosure is to provide a composite medical balloon including an outer layer and an inner layer comprising different materials, and a shaft comprising a material compatible (meaning capable of being thermally bonded so as to form a fluid-tight seal) with the inner layer of the balloon. In this manner, the balloon may be formed using coextrusion techniques and welded directly to the shaft, while the balloon outer layer may provide different characteristics given the lack of any need for shaft compatibility. The use of adhesives for forming any joints between the catheter and shaft may thus be avoided, and enhanced flexibility may result, along with an ease of manufacturing and validation.

According to a first aspect of the disclosure, an apparatus for performing a medical procedure is provided. The apparatus comprises a coextruded inflatable balloon having an outer layer and an inner layer comprising different materials. A shaft comprises a material compatible with the inner layer of the balloon and welded thereto so as to form a balloon catheter.

In one embodiment, the inflatable balloon further includes an intermediate layer for bonding the inner layer to the outer layer. The intermediate layer may comprises polyolefin.

In this or another embodiment, the outer layer comprises polyester and the inner layer comprises polyamide. In one example, the polyester comprises PET and the polyamide comprises nylon. In another example, the material of the shaft comprises polyamide.

A weld may serve to connect the material of the shaft to the inner layer of the inflatable balloon. In such an example, the shaft is not connected to the balloon using an adhesive. To facilitate welding, the inner layer and the shaft comprise the same material.

In another aspect of the disclosure, an apparatus for performing a medical procedure is provided. The apparatus comprises an inflatable balloon comprising an outer layer comprising a polyester and an inner layer comprising a polyamide.

In one embodiment, an intermediate layer for bonding the inner layer to the outer layer is also provided. The polyester may comprise PET and the polyamide may comprise nylon. The inflatable balloon may be coextruded, and may be welded to a shaft comprising a material compatible with the inner layer of the inflatable balloon (such as for example, a polyamide in a case where the shaft comprises nylon).

Still a further aspect of the disclosure pertains to an apparatus for performing a medical procedure. The apparatus comprises an inflatable balloon comprising a polyester and a shaft comprising a polyester connected to the inflatable balloon.

In one embodiment, the polyester comprises an inner layer of the inflatable balloon, and further comprising an outer layer comprising a material different from polyester. An intermediate layer may be provided for bonding the inner and outer layers. A weld may be provided for bonding the shaft to the inflatable balloon.

Yet a further aspect of the disclosure relates to a method of forming a medical balloon including a shaft. The method comprises coextruding the medical balloon including an outer layer and an inner layer compatible for welding with the material of the shaft, the outer layer and the inner layer comprising different materials. The method further comprises welding the inner layer of the medical balloon to the shaft.

In one embodiment, the coextruding step comprises providing an intermediate layer for bonding the outer layer and the inner layer. The outer layer of the balloon may comprise polyester and the inner layer may comprise polyamide. More specifically, the outer layer may comprise PET and the inner layer may comprise nylon.

### BRIEF DESCRIPTION OF THE DRAWINGS

The above and further advantages of the disclosure may be better understood by referring to the following description in conjunction with the accompanying drawings in which:

FIG. 1 illustrates a semi-cross section of a medical balloon;

FIG. 2 schematically illustrates a coextruder for forming a multi-layered parison;

FIG. 3. illustrates a partial view of a multi-layered parison for forming into a balloon;

FIG. 4 illustrates the parison being blow-molded into a balloon; and

FIG. 5 illustrates the balloon being thermally welded to a shaft.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

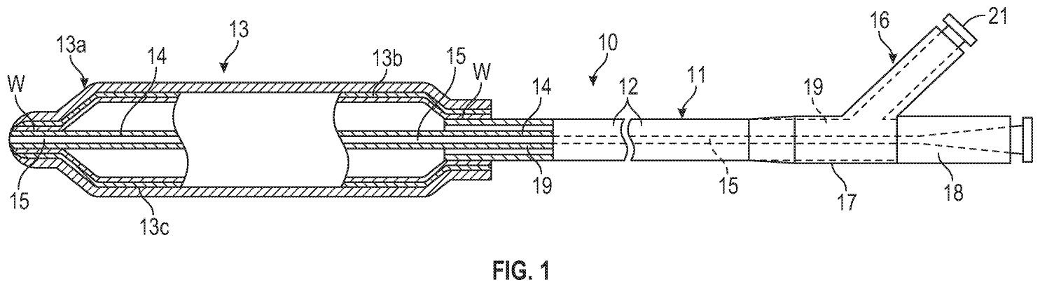

With reference to FIG. 1, a partial cross section of a catheter 10 is shown, which includes a composite inflatable balloon 13 made from layers of different materials, according to the following description. As can be understood, the balloon catheter 10 shown in FIG. 1 includes an elongated shaft 11 and a balloon 13. The shaft 11 may comprise an inner tube 14 and an outer tube 12. The balloon 13 is disposed on the distal end portion of the shaft 11.

The inner tube 14 has an inner lumen 15 opened at the distal end of the inner tube 14, such as for receiving a guidewire (not shown). The outer tube 12, provided coaxially with the inner tube 14, has a distal end on the proximal side from the distal end of the inner tube 14 by a specific length. In the illustrated embodiment, the balloon 13 is fixed to both a distal end portion of the inner tube 14 and a distal end portion of the outer tube 12. While this coaxial arrangement of tubes 12, 14 is often used, the catheter 10 may comprise a single tube as the shaft 11 in certain applications, or more than two tubes in others, and the construction of the shaft 11 is not considered a limitation on the disclosed invention.

The inner volume of the balloon 13 is inflatable or deflatable by injecting or removing a fluid in or from the balloon 13. The fluid may be supplied or withdrawn by the indeflator (not shown), which may be connected to a branch hub 16 having an outer tube hub 17 and an inner tube hub 18. The outer tube hub 17 is sealed to a proximal end portion of the outer tube 12, and has an opening portion 21 communicating with a lumen 19 of the outer tube 12. The opening portion 21 functions as an inlet or outlet through which the fluid is to flow in or from the balloon 13 for inflating or deflating the balloon 13.

According to one aspect of the disclosure, the balloon 13 comprises multiple layers of different materials, such as two different polymer compounds. In one particular example, the balloon 13 may include an outer layer 13a and an inner layer 13b comprising different materials in order to improve the functional characteristics of the balloon and resulting catheter 10 formed when the balloon 13 is joined to a shaft 11. More specifically, the outer layer 13a (which is shown as the outermost later) may comprise a material that is tougher or more resilient, while the inner layer 13b (which is shown as the innermost layer) comprises a material compatible (meaning capable of being thermally bonded so as to form a fluid-tight seal) with the material used to form the shaft 11, so as to allow for secure bonding without a requirement for using adhesives.

In one example, the outer layer 13a comprises polyester and the inner layer 13b comprises polyamide, in which case the shaft 11 or, in particular, the outer tube 12 and the inner tube 14 may comprise a polyamide as well. More specifically, the polyester forming the outer layer 13a comprises polyethylene terephthalate, or PET, and the polyamide of the inner layer 13b comprises nylon (such as for example, Nylon 12). The outer tube 12 and the inner tube 14 forming the shaft 11 may also comprise nylon (such as in the form of the PEBAX material distributed by the Arkema Group in one particular example). The PET or polyester is considered to be more resilient or tougher, and thus desirable for use as an outer layer, whereas, the nylon inner layer is fully compatible with the typical shaft material, and thus may be bonded to it via thermal bonding.

In another example, the shaft 11 and in particular tubes 12, 14, and the inner layer 13b of the balloon 13 may both comprise polyester materials, such as for example PET, and the outer layer 13a may comprise either a polyester, such as PET, or a different material, such as nylon. In the former case, the shaft 11 and balloon 13 would thus be made of homogenous materials, which may reduce manufacturing complexity and cost, and may also facilitate recycling. Additional layers may also be provided as long as the desirable objectives of material compatibility according to the disclosure are achieved.

As a result of using compatible materials for the inner layer 13b of the balloon 13 and the shaft 11, or outer tube 14 and inner tube 12 in particular, assembly of the two structures can be achieved without the need for using an adhesive at the joint thus formed. For example, thermal bonding or welding may be used to bond the inner layer 13b to these tubes 12, 14. This may be done in one example by forming a circumferential weld W at the corresponding proximal and distal points of contact established between the balloon 13 and the tubes 12, 14, if present.

In order to achieve bonding of the dissimilar materials forming the outer layer 13a and inner layer 13b of the balloon 13, an intermediate layer 13c may be provided for fixing or bonding the inner layer 13b to the outer layer 13a. This intermediate layer 13c may comprise, for example, a synthetic resin used for bonding, such as polyolefin, and thus provides an adhesive or tie layer to interconnect the outer layer 13a and the inner layer 13b. One particular example of such a resin is PLEXAR anhydride-modified polyolefin resin distributed by LyondellBasell, and another is an OREVAC is a maleic anhydride modified ethylene methyl-acrylate (EMA) copolymer resin distributed by the Arkema Group (such as, for example OREVAC 18603).

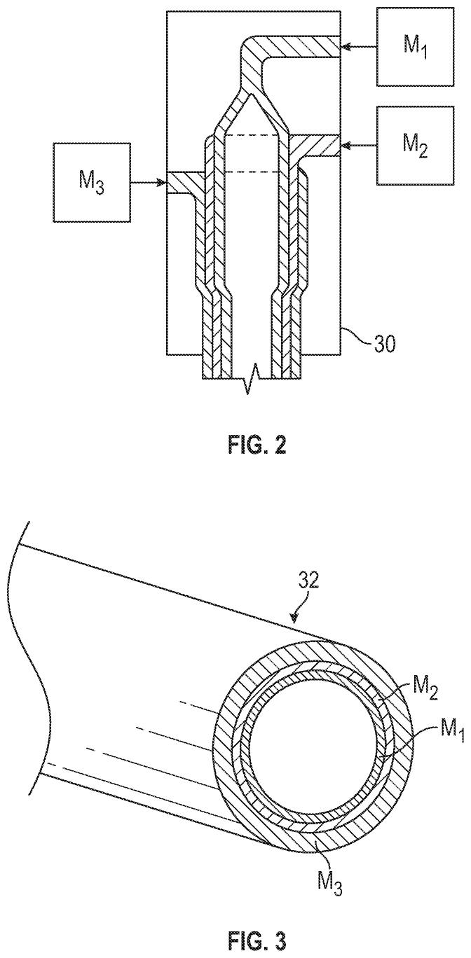

Formation of the medical balloon 13 according to the disclosure may be achieved using a coextrusion technique. This technique or process may involve concurrently feeding the materials for forming the various layers. For example, as shown in FIG. 2, three different polymer materials M1, M2, M3 may be coextruded for forming the inner, outer, and intermediate layers 13a, 13b, 13c of the balloon 13, such as by being fed into a coextruder. The coextruder may comprise a coextrusion die 30, adapted to form a laminated tubular parison 32, as shown in FIG. 3, comprising the three (or more) materials M1, M2, and M3 as interbonded layers.

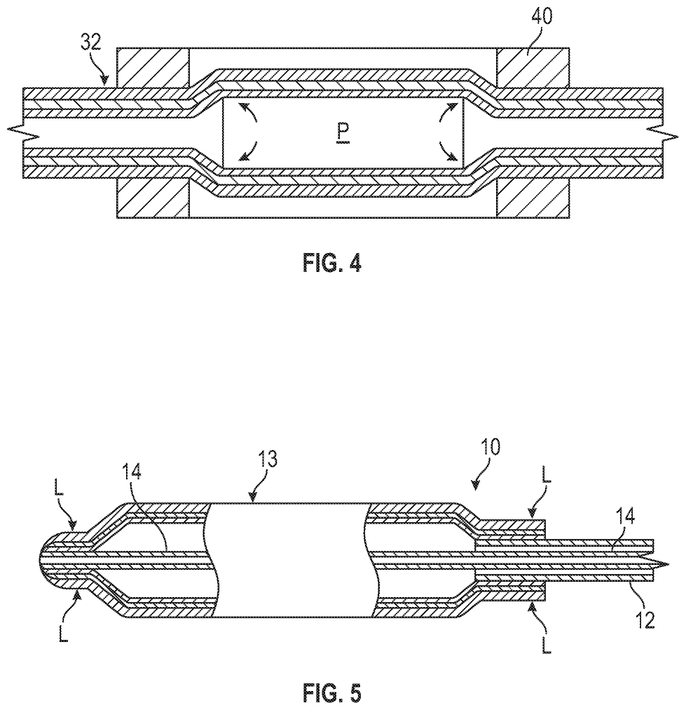

As shown in FIG. 4, the parison 32 may then be formed into a suitable inflatable balloon 13 for catheter 10 using known blow molding techniques, such as using a suitable die 40 and with the application of internal pressure P. The blow-molded balloon 13 may then attached to the shaft 11 via thermal bolding, such as by using one or more lasers L, to weld the outer tube 12 and the inner tube 14 to the inner layer 13b of the balloon, as noted above, in order to form the catheter 10. Accordingly, at least the distal portion of the balloon catheter 10 may be formed without any requirement for adhesives to form bonds at joint locations, which as noted above may provide certain advantages.

If desired to achieve a particular objective, fibers could also be added to the balloon 13. For example, fibers could be added during the coextrusion process, or applied later once the balloon 13 is formed by application to the outer layer 13a or inner layer 13b. The fibers may extend longitudinally, helically, or both, and may be provided in one or more layers, together with an adhesive, and possibly an outermost film layer to provide the balloon 13 with a smooth continuous outer surface.

Summarizing, this disclosure may be considered to relate to the following items:
1. An apparatus for performing a medical procedure, comprising:
   a coextruded inflatable balloon having an outer layer and an inner layer comprising different materials, such as different polymer compounds; and
   a shaft comprising a material compatible with the inner layer of the balloon for purposes of being welded thereto, such that the use of adhesives to form a joint may be avoided.
2. The apparatus of item 1, wherein the inflatable balloon further includes an intermediate layer for bonding the inner layer to the outer layer.
3. The apparatus of item 2, wherein the intermediate layer comprises polyolefin.
4. The apparatus of any of items 1-3, wherein the outer layer comprises polyester and the inner layer comprises polyamide.
5. The apparatus of item 4, wherein the polyester comprises PET and the polyamide comprises nylon.
6. The apparatus of any of items 1-5, wherein the material of the shaft comprises polyamide.
7. The apparatus of any of items 1-6, further including a weld connecting the material of the shaft to the inner layer of the inflatable balloon.
8. The apparatus of any of items 1-7, wherein the shaft is not connected to the balloon using an adhesive.
9. The apparatus of any of items 1-8, wherein the inner layer and the shaft comprise the same material.
10. An apparatus for performing a medical procedure, comprising:
   an inflatable balloon having an outer layer comprising a polyester and an inner layer comprising a polyamide.
11. The apparatus of item 10, further including an intermediate layer for bonding the inner layer to the outer layer.
12. The apparatus of item 10 or item 11, wherein the polyester comprises PET and the polyamide comprises nylon.
13. The apparatus of any of items 10-12, wherein the inflatable balloon is coextruded.
14. The apparatus of any of items 10-13, further including a shaft welded to the inflatable balloon.
15. The apparatus of item 14, wherein the shaft comprises a material compatible with the inner layer of the inflatable balloon.
16. The apparatus of item 15, wherein the material of the shaft comprises a polyamide
17. The apparatus of item 14 or item 15, wherein the material of the shaft comprises nylon.
18. An apparatus for performing a medical procedure, comprising:
   an inflatable balloon comprising a polyester; and
   a shaft comprising a polyester connected to the inflatable balloon.
19. The apparatus of item 18, wherein the polyester comprises an inner layer of the inflatable balloon, and further comprising an outer layer comprising a material different from polyester.
20. The apparatus of item 19, further including an intermediate layer for bonding the inner and outer layers, such as via coextrusion or adhesive bonding.
21. The apparatus of any of items 18-20, further including a weld for bonding the polyester of the shaft to polyester of the inflatable balloon.
22. A method of forming a medical balloon including a shaft, comprising:
   coextruding the medical balloon including an outer layer and an inner layer compatible for welding with the material of the shaft, the outer layer and the inner layer comprising different materials; and
   welding the inner layer of the medical balloon to the shaft.
23. The method of item 22, wherein the coextruding step comprises providing an intermediate layer for bonding the outer layer and the inner layer.
24. The method of item 22 or item 23, wherein the outer layer comprises polyester and the inner layer comprises polyamide.
25. The method of any of items 22-24, wherein the outer layer comprises PET and the inner layer comprises nylon.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. For example, while certain polymers are mentioned, it should be appreciated that others could be successfully used while achieving the desired benefits (such as, for instance, polyfuranoates (and in particular poly(ethylene-2,5-furanoate) (PEF)) in the place of PET). Furthermore, while a tri-layer extrusion is illustrated, additional layers could be provided (such as for example a further outer polyamide layer, which could be functionally modified (e.g., made hydrophilic) for use with coatings, such as treatment agents or drugs), along with an associated tie layer. While coextrusion is mentioned, adhesives or resins applied to the layers of the balloon in other ways may also be used. The mention of an outer layer does not necessarily mean it is the outermost layer, and likewise, the mention of an inner layer does not necessarily mean it is the innermost layer. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. An apparatus for performing a medical procedure, comprising:
a coextruded inflatable balloon having an outer layer and an inner layer comprising different materials; and
a shaft comprising a material compatible with the inner layer of the balloon and welded thereto.

2. The apparatus of claim 1, wherein the inflatable balloon further includes an intermediate layer for bonding the inner layer to the outer layer.

3. The apparatus of claim 2, wherein the intermediate layer comprises polyolefin.

4. The apparatus of claim 1, wherein the outer layer comprises polyester and the inner layer comprises polyamide.

5. The apparatus of claim 4, wherein the polyester comprises PET and the polyamide comprises nylon.

6. The apparatus of claim 1, wherein the material of the shaft comprises polyamide.

7. The apparatus of claim 1, further including a weld connecting the material of the shaft to the inner layer of the inflatable balloon.

8. The apparatus of claim 1, wherein t nected to the balloon using an adhesive.

9. The apparatus of claim 1, wherein the inner layer and the shaft comprise the same material.

10. An apparatus for performing a medical procedure, comprising:
an inflatable balloon having an outer layer comprising a polyester and an inner layer comprising a polyamide.

11. The apparatus of claim 10, further including an intermediate layer for bonding the inner layer to the outer layer.

12. The apparatus of claim 10, wherein the polyester comprises PET and the polyamide comprises nylon.

13. The apparatus of claim 10, wherein the inflatable balloon is coextruded.

14. The apparatus of claim 10, further including a shaft welded to the inflatable balloon.

15. The apparatus of claim 14, wherein the shaft comprises a material compatible for welding with the inner layer of the inflatable balloon.

16. The apparatus of claim 15, wherein the material of the shaft comprises a polyamide.

17. The apparatus of claim 15, wherein the material of the shaft comprises nylon.

18. An apparatus for performing a medical procedure, comprising:
an inflatable balloon comprising a polyester; and
a shaft comprising a polyester connected to the inflatable balloon.

19. The apparatus of claim 18, wherein the inflatable balloon comprises an inner layer comprising the polyester, and further comprising an outer layer comprising a material different from polyester.

20. The apparatus of claim 19, further including an intermediate layer for bonding the inner and outer layers.

21. The apparatus of claim 18, further including a weld for bonding the shaft to the inflatable balloon.

22. A method of forming a medical balloon including a shaft, comprising:
coextruding the medical balloon including an outer layer and an inner layer compatible for welding with the material of the shaft, the outer layer and the inner layer comprising different materials; and
welding the inner layer of the medical balloon to the shaft.

23. The method of claim 22, wherein tl comprises providing an intermediate layer for bonding the outer layer and the inner layer.

24. The method of claim 22, wherein the outer layer comprises polyester and the inner layer comprises polyamide.

25. The method of claim 24, wherein the outer layer comprises PET and the inner layer comprises nylon.
